(19) United States
(12) Patent Application Publication
(10) Pub. No.: US 2004/0052824 A1
(43) Pub. Date: Mar. 18, 2004

(54) Micellar colloidal pharmaceutical composition containing a lipophilic active principle

(76) Inventors: Marie-Line Abou Chacra-Vernet, Nice (FR); Claude Laruelle, Villeneuve-Loubet (FR); Dominique Toselli, Nice (FR)

Correspondence Address: Thomas W Tolpin, Welsh & Katz, 22nd Floor, 120 South Riverside Plaza, Chicago, IL, 60606, US

(21) Appl. No.: 10/465,923

(22) PCT Filed: Dec. 27, 2001
(86) PCT No.: PCT/FR01/04212

(30) Foreign Application Priority Data
Dec. 28, 2000 (FR) 00/17250

(51) Int. Cl. A61K 31/56; A61K 31/203; A61K 35/78; A61K 9/00

(52) U.S. Cl. 424/400; 424/725; 514/171; 514/559

(57) ABSTRACT

The invention concerns novel pharmaceutical compositions capable of comprising micelles containing at least a very lipophilic principle, enabling to enhance bioavailability of active principles insoluble in aqueous solvents called MIDDS® (Micellar Improved Drug Delivery Solutions).

The present invention relates to novel micelle-forming pharmaceutical compositions containing at least one lipophilic active principle, which make it possible to increase the bioavailability of active principles insoluble in aqueous solvents, designated by the term MIDDS® (Micellar Improved Drug Delivery Solution).

This novel micelle-forming pharmaceutical dosage form is similar to known forms described as Self Emulsifying Drug Delivery Systems (SEDDS) or self-emulsifying systems, comprising a lipid phase and large quantities of surfactants (S) and/or of solvents. These self-emulsifying systems have been used for a long time in pharmaceutical preparations.

In the pharmaceutical industry, improving the bioavailability of very lipophilic active principles (AP) intended to be administered orally is of great interest to formulation pharmacists.

The formulation of lipophilic APs, and a fortiori that of very lipophilic APs, poses real problems mainly due to their low solubility in aqueous liquid pharmaceutical excipients, to their propensity to precipitate or to recrystallize from aqueous solution and to their low solubility in the fluids of the gastrointestinal tract from which they have to be absorbed.

Various techniques have already been proposed which are aimed at improving the solubilization of these lipophilic (hydrophobic) APs and their absorption from the digestive system, in order to increase the bioavailability thereof.

The improvement of the therapeutic efficacy of hydrophobic APs by virtue of their formulation as an oily solution or with the aid of their administration after a meal high in lipids has been exploited for several decades.

The bioavailability of an AP depends on its concentration in the gastrointestinal fluid; the latter itself depends on the release of the AP from the oily phase. The more lipophilic an AP, the less it tends to migrate in the digestive fluids. The absorption of these oily solutions starts by a hydrolysis at the oil-water interface, followed by a solubilization in the micelles of bile salts which penetrate into the intestinal microvilli, thus transporting the hydrophobic AP (NA Armstrong et al., Int. J. Pharm., 1980, 6, 195-204).

The release of an AP from an oily formulation provided in appropriate gelatin capsules is common and numerous pharmaceutical preparations are marketed in this form.

Accordingly, French patent application FR-A-2 408 345 describes the preparation of micronized progesterone which is provided in the form of an oily suspension thereof, in particular based on vegetable oil (soybean lecithin, peanut oil, etc) in soft gelatin capsules. This proprietary product is marketed under the name Utrogestan®.

It is also known that the bioavailability of lipophilic APs can be increased by their formulation with the aid of digestible oils and hydrophilic and lipophilic surfactants (K J McGREGOR et al., Adv. Drug Deliv. Rev., 1997, 25, 33-46 and international application WO 95/24893). This type of formulation makes it possible to maintain the AP in solution during its passage into the digestive tract and until its intestinal absorption.

The digestion of oil ingredients of this type of formulations often has the advantage of solubilizing the AP in mixed micelles consisting of bile salts and products of lipolysis of the triglycerides of the digestible oil used.

However, the presence of surfactants can inhibit lipolysis, which requires the preliminary evaluation in vitro of the digestibility of the oils of a given formulation. Moreover, the quantities of digestible oils which should sometimes be used in order to avoid the recrystallization of the AP in vivo are too high to allow the manufacture of a marketable capsule.

It has, moreover, already been envisaged to optimize the lipid formulation of scarcely soluble APs using self-emulsifying vehicles capable of spontaneously forming a microemulsion upon contact with an aqueous phase in vitro and, likewise, at the level of the site of absorption in vivo.

These self-emulsifying preparations (Self-Emulsifying Drug Delivery Systems: SEDDS) are mixtures of oils and surfactants, which are isotropic, sometimes containing cosolvents and which self-emulsify with gentle stirring, a condition similar to the conditions encountered in the digestive tract (C W Pouton, Int. J. Pharm., 1985, 27, 335-348; M G Wakerly et al., Am. Chem. Soc. Symposium Series, 1986, 311, 242-255; Charman et al., Pharm. Res., 1992, 9, 87-93; B J Aungst, J. Pharm. Sci., 1993, 82, 979-987; P Constantinides, Pharm. Res., 1995, 12, 1561-1572).

The physical phenomena which explain the formation of microemulsions and the equilibria governing them have been widely studied and modelled (M Borkovec, Adv. Colloid Interface Sci., 1992, 37, 195-217 and references cited).

Microemulsions find numerous applications in various fields. The study of the formulation of these transparent dispersions has allowed researchers interested by their physicochemical potentials (H L Rosano et al., J. Colloid Interface Sci., 1979, 72, 233-244) or pharmaceutical potentials (C W Pouton, mentioned above, W A Ritschel et al., Meth. Find. Exp. Clin. Pharmacol., 1990, 12, 127-134 and W A Ritschel, Meth. Find. Exp. Clin. Pharmacol., 1991, 13, 205-220) to specify the relevant procedures and the appropriate methods for analyzing the performance of the so-called SEDDS self-emulsifying systems.

More particularly, patent application EP-A-0 670 715 describes SMEDDS® (Self Micro-Emulsifying Drug Delivery System) which contain a lipophilic active principle such as indometacin, diclofenac and hydrocortisone, a lipophilic phase preferably representing from 10 to 75% by weight of the total weight of the composition and consisting of a mixture of glycerides and C₈-C₁₈ fatty acid esters having a hydrophilic-lipophilic balance (HLB) of less than 16 and preferably of close to 14, a surfactant based on glycerides having an HLB of less than 16, a cosurfactant (CoS) chosen from lauryl esters of propylene glycol, oleyl esters of polyglycerol and ethyl diglycol, the S/CoS ratio being between 0.5 and 6. Upon contact with hydrophilic phase, consisting for example of the physiological fluid of the intestinal medium, this composition spontaneously forms a microemulsion.

These microemulsifying systems make it possible to solubilize certain hydrophobic APs; however, they do not make it possible to systematically improve their bioavailability (Farah, self-microemulsifying drug delivery systems for improving in-vitro dissolution of drugs: AAPS Annual meeting Orlando, Fla., 1993).

Now, maintaining a lipophilic AP in micellar solution allowing its intestinal absorption is the key to success in preparing an effective lipid formulation.

Furthermore, the best SEDDSs, i.e. those which solubilize a large quantity of AP and which form very fine micellar dispersions, are generally the most hydrophilic. Now, it is for these hydrophilic SEDDSs (containing a hydrophilic S and CoS having high HLB values, in general greater than 12) that the risks of recrystallization of the AP in vivo are the greatest (Pouton, Bulletin Technique Gattefossé, 1999, 92, 41-49) and consequently the suprabioavailability of the AP is not necessarily achieved.

Indeed, while this type of self-micro emulsifying systems, which is described in patent application EP-A-0 670 715 (with a lipophilic phase having an HLB of close to 14), makes it possible to improve the formulation and the bioavailability of certain APs, it leads, on the other hand, with very lipophilic APs, to unstable lipid medicinal solutions and, for some extremely lipophilic active principles, this physical instability of the lipid medicinal solution is additionally coupled with the formation of unstable microemulsions upon contact with an aqueous phase, hence a failure of the pharmaceutical formulation.

Moreover, international application WO 96/21439 describes formulations based on a mixture of saturated polyglycolized C₈-C₁₈ glycerides having an HLB=14 (Gelucire®—company Gattefosse) and fenofibrate which is a lipophilic AP.

However, in order to be stable, these formulations require the presence of a cellulosic polymer so as to increase the viscosity thereof.

Finally, there has also already been proposed, in particular in international application WO 99/56727, the formulation of active principles which are slightly soluble in water by means of self-emulsifying compositions, of emulsions or of microemulsions containing from 5 to 70% of an oily component having an HLB of less than or equal to 4 and a surfactant system containing one or more surfactants having an HLB of between 10 and 20; these compositions being substantially free of hydrophilic solvent system.

While this type of compositions has improved stability properties given the absence of a hydrophilic solvent system, it is not satisfactory for solubilizing very lipophilic active principles.

The lipophilicity of an AP can be determined as a function of its coefficient of partition (P) between octanol and water which corresponds to the concentration of the AP in octanol (C_{Oct})/concentration of the AP in water (C_{Water}) ratio.

The determination of the partition coefficient is a factor which is widely exploited in various fields of application of therapeutic or pharmacochemical chemistry, from the synthesis of chemical substances for medicinal use to the analysis of pharmaceutical products.

This characteristic is in particular taken into consideration by pharmacologists and toxicologists given the fundamental importance of the partition of medicinal active principles between biological media (in particular in terms of absorption and distribution) for the expression of their activity and/or their toxicity.

Thus, the determination of the octanol/water partition (P) coefficient, generally expressed as log P, is a major factor among the indicators of the structure-activity relationships for medicinal active principles or for toxic substances (C. Hansch et al., Exploring QSAR, (1995), Vol. I & II, Ed. American Chemical Society, USA; C. Hansch et al., J. Pharm. Sci., 1987, 76, 663-687; V. Pli\check{s}ka et al., Lipophilicity in drug action on toxicology, Vol. 4 Verlagsgesellschaft mbH. Weinheim (1996); H. van de Waterbeemd, Quantitative approaches to structure-activity relationships, in: The practice of medicinal chemistry, Ed.: Wermuth, Academic Press, London (1996); Association Francaise des Enseignants de Chimie Thérapeutique: Traité de Chimie Thérapeutique, 7 volumes, Ed. TEC & DOC. Paris, (1992-2000).

When the P ratio is greater than 1, it means that C_{Oct.} is greater than C_{Wat.} and that consequently the AP is lipophilic (log P>0). It is therefore possible to deduce therefrom that the higher the log P of an AP, the more pronounced the lipophilic character exhibited by it.

However, this physicochemical characteristic is not used in the field of galenic pharmacy (pharmaceutical formulation) and no prior art document makes reference to the notion of log P and really takes into account this criterion in the formulation strategy.

It is in order to overcome all these problems that the inventors have developed that which constitutes the subject of the invention.

The inventors set themselves the objective of providing a self-emulsifying pharmaceutical composition intended for oral administration, capable of forming a micellar solution or a microemulsion upon contact with digestive fluids, thus allowing the formulation of very lipophilic, or even extremely lipophilic, active principles while improving their bioavailability, said composition being stable in the liquid state and in the form of a microemulsion and leads to a very fine and homogeneous micellar dispersion.

For the purposes of the present invention, it is considered that the very lipophilic APs are those having a log P greater than 2, the extremely lipophilic APs having a log P greater than 4.

The subject of the present invention is therefore a self-micro emulsifying pharmaceutical composition for oral use comprising:

at least one lipophilic active principle,

at least one surfactant having a hydrophilic/lipophilic balance of less than 16,

at least one cosurfactant,

at least one lipophilic phase, characterized in that:

the lipophilic active principle(s) have a log P greater than 2,

the surfactant(s) represent at least 50% by weight of the total weight of said composition,

the cosurfactant(s) are chosen from the good solvents for said active principle(s),

the lipophilic phase is optionally surface-active and represents from 0.5 to 4.5% of the total weight of said composition and has an HLB of less than or equal to 6, and

when the active principle is different from a retinoid, then said composition additionally comprises a nonsurfactant oily phase representing from 1 to 12% of the total weight of said composition.

The pharmaceutical composition in accordance with the invention is essentially distinguishable from those described by the prior art by the fact that the lipophilic phase and the oily phase have very low HLB values and are used in small quantities and also by the essential presence of a CoS which has a role of good solvent for the AP in the pharmaceutical dosage form.

The inventors have indeed demonstrated that this composition allows the dissolution of very lipophilic APs and leads, in the presence of a hydrophilic phase, to formulations forming fine, stable and homogeneous micellar colloidal dispersions, thus making it possible to improve the bioavailability of these APs in the gastrointestinal tract.

The pharmaceutical composition in accordance with the invention makes it possible in particular to obtain microemulsions whose micelles have a size of less than 500 nm and more particularly of between 1 and 200 nm.

Depending on the excipients used in their formulation, there may be liquid lipid solutions or solid (semisolid, pasty) solutions at room temperature. The pharmaceutical compositions in accordance with the present invention form in all cases a microemuision or a colloidal solution, of the micellar type, upon contact with an aqueous phase.

All the APs having a log P greater than 2, and more particularly greater than 4, may be used in accordance with the present invention.

These very lipophilic, or even extremely lipophilic, active principles are generally molecules having long carbon chains and/or aromatic rings or nuclei carrying hydrophobic substituents, with very few hydrophilic groups or substituents.

These very lipophilic active principles may be chosen in particular from retinoids, hypolipidemic agents, steroid hormones, steroid anti-inflammatories, nonsteroid anti-inflammatories (NSAIDs), antiretrovirals, protease inhibitors (“navirs”), antiacids, proton pump inhibitors, antiemetics, fat-soluble vitamins, cardiovascular system drugs, platelet aggregation inhibitors, cancer drugs, certain plant extracts and their isolated or derived APs, immunosuppressants, central nervous system drugs, antimigraines, antibiotics, antifungals and antiparasitics, provided of course that they have a log P greater than 2.

Retinoids are compounds capable of binding and interacting with a retinoic acid receptor (alpha, beta or gamma RAR) or with a retinoid X receptor (alpha, beta, gamma RXR). As examples of such retinoids, there may be mentioned, firstly, the retinoids derived from vitamin A such as tretinoin, also known by the name of all-trans-retinoic acid or all-trans-vitamin A acid, isotretinoin which corresponds to the 13-cis isomer of tretinoin, and which, as a result, is also called 13-cis-retinoic acid or 13-cis vitamin A acid, 9-cis-retinoic acid or 9-cis vitamin A acid, acitretin, etretinate, but also the acetylene retinoids such as tazarotene, the retinoids derived from naphthalene such as lonapalene and 2-(5,6,+8-tetrahydromethyl-2-anthryl)-4-thiophenocarboxylic acid, and the retinoids containing an adamantyl ring such as adapalene, 6-[3-(1-adamantyl)-4-hydroxyphenyl]-2-naphthoic acid and 4-[3-(1-adamantyl)-4-methoxybenzamido]benzoic acid and their esters.

Among these retinoids, the use of isotretinoin (log P=6) is particularly preferred according to the invention.

As hypolipidemic agents, which are compounds capable of inhibiting the synthesis of cholesterol and triglycerides, there may be mentioned, in particular, fibrates such as 1-methylethyl ester of 2-(4-(4-chlorobenzoyl)phenoxy)-2-methylpropanoic acid also called fenofibrate (log P=5.24) and related products of fibrate class such as clofibrate (log P=3.65), bezafibrate (log P=3.53), ciprofibrate (log P=3.15) and gemfibrozil (log P=3.90).

There may also be mentioned, among the other hypolipidemic agents, bisthioethers, including probucol and tiadenol (log P=4.58), the class of HMG Co-A reductase inhibitors (statins) such as for example simvastatin (log P=4.68), mevastatin (log P=3.52), lovastatin (log P=4.04), atorvastatin, pravastatin, fluvastatin, cerivastatin, and the class of ACAT inhibitors such as melinamide and its structural analogs.

As steroid hormones, there may be mentioned in particular estrogens which are estradiol derivatives and esters of (log P>5), progesterone (log P=3.87), danazol (log P=4.53), testosterone (log P=3.32) and esters and derivatives of testosterone (log P>4). There may also be mentioned antiandrogens including flutamide (log P=3.5), nilutamide; 5α-reductase inhibitors, competitive inhibitors of testosterone such as finasteride (log P=3.03); quinazoline derivatives such as alfuzosin; nonsteroid agonists/antagonists of the estrogen receptors such as tamoxifen (log P=4.03) and raloxifen.

As steroid anti-inflammatories, there may be mentioned in particular glucocorticoids having a log P of between 2 and 3 such as prednisolone, cortisone, its esters and derivatives (log P=2.1 to 2.4).

As NSAIDs, there may be mentioned in particular mefenamic acid (log P=5.3), naproxen (log P=2.90), nabumetone (log P=3 32), ibuprofen (log P=3.50 to 4.50) and COX-2 inhibitors such as celecoxib, rofecoxib, parecoxib and valdecoxib.

Antiretrovirals and protease inhibitors are compounds which are sparingly soluble in water, whose partition coefficients can be calculated or determined by the analytical route, among which there may be mentioned amprenavir (solubility 0.04 mg/l), saquinavir and saquinavir mesylate (solubility 2.22 mg/ml) and ritonavir (almost insoluble in water).

As antiacids and proton pump inhibitors, there may be mentioned in particular omeprazole (log P=2.23), pantoprazole, rabeprazole (or pariprazole), lansoprazole and timoprazole.

As antiemetics, there may be mentioned in particular domperidone (log P=4.05), serotonin antagonists (“setrons”) such as ondansetron (log P=2.63), granisetron and azasetron.

As fat-soluble vitamins, there may be mentioned in particular vitamins A or retinol (log P=5.68), D including calcitriol, E or tocopherols, K or menadione (log P=8).

Among the cardiovascular system drugs, there may be mentioned in particular antagonists of angiotensin II (sartans) such as valsartan, losartan, irbesartan, candesartan, tasosartan, telmisartan (log P=4.8); α- and β-blockers such as carvediol, celiprolol (log P=2.07); calcium inhibitors (dihydropyridines) such as verapamil (log P=3.8), diltiazem (log P=2.7), nifedipine (log P=2.75) and nitrendipine (log P=3.7). It is also possible to mention other compounds, antihypertensives, such as renin-inhibiting peptides, oxazolidinone derivatives or glycol peptides substituted with amino residues and/or azole- or thiazole-containing heterocyclic rings (log P of between 2 and 4).

As platelet aggregation inhibitors, there may be mentioned in particular clopidogrel (oil), ticlopidine; coumarinic anticoagulants including warfarin (log P=2.70) and compounds of the indanedione group, including phenyl indanedione (log P=2.90).

As cancer drugs, there may be mentioned in particular paclitaxel and docetaxel which are compounds which are insoluble in water; Vinca minor extracts and alkaloids such as vincristine (log P=2.80), vincaleukoblastin or vinblastin (log P=3.69), vincamine and their derivatives; Ochrosia elliptica alkaloids including ellipticine (log P=4.80).

Among the plant extracts and their isolated or derived APs, there may be mentioned in particular alkaloids such as yohimbine (log P=2.73), flavonoids including diosmin, rutin and its derivatives such as troxerutin; Pygeum africanum or Serenoa repens extracts.

As immunosuppressants, there may be mentioned in particular ciclosporin (log P=2.92) and tacrolimus.

Among the various central nervous system drugs are tranquilizers, sedatives, hypnotics and anesthetics. By way of example, there may be mentioned barbiturates (log P of between 2 and 2.5) such as thiobarbiturates (log P of close to 3); anxiolytics such as benzodiazepines (log P of between 2 and 3); antihistamines (log P of between 2 and 5) such as terfenadine (log P=3.22), loratadine (log P=5.20), desloratadine and cetirizine; tricyclic and serotoninergic antidepressants such as fluoxetine, paroxetine, sertraline and citalopram.

As antimigraines, there may be mentioned compounds of the serotoninergic “triptans” group such as oxitriptan, sumatriptan and almotriptan.

Among the antibiotics, there may be mentioned in particular third generation cephalosporins such as cefixim trihydrate and cefpodoxim proxetil; macrolides such as azithromycin, clarithromycin, roxithromycin (log P of close to 2.5), josamycin (log P=2.39), spiramycin; synergistins such as pristinamycin, quinolones and quinoxalines, including carbadox.

Among the antifungals, there may be mentioned in particular griseofulvin (log P=2.18), amphotericin B, terbinafin (log P=5.42) and azole-containing antifungals (conazoles) including miconazole (log P=2.3 and 5.6), itraconazole (log P=5.68), ketoconazole (log P=4.34) and fluconazole.

Among the antiparasitics, there may be mentioned antimalarials such as halofantrine (log P=8.2), mefloquin (log P=3.36), proguanil (log P=2.53), pyrimethamine (log P=2.69), extracts of Artemisia spp and substances isolated from these extracts and their derivatives such as artemisin, artemisinin and their derivatives (log P=2.2 to 4); the avermectin series, including ivermectin which is practically insoluble in water (partition coefficient chloroform/water: log P=3; partition coefficient ethyl acetate/water: log P=4); anthelminthics derived from benzimidazole for veterinary use such as for example tiabendazole (log P=2.31), albendazole (log P=3.22), mebendazole (log P=3.10), fenbendazole (log P=4.26) and triclabendazole (log P=6.45); the class of salicylanilides for veterinary use, used in fascioloses (douvicides) and other parasitoses comprising in particular bromoxanide (log P=5.65), brotianide (log P=5.30), clioxanide (log P=5.45), closantel (log P>7), oxyclozanide (log P=5.35), rafoxanide (log P=8.75) and dibromosalan (log P=5.18) and tribromosalan (log P=5.86).

According to the invention, the active principle(s) are preferably chosen from retinoides, hypolipidemic agents and steroid hormones.

According to the invention, the active principle(s) having a log P greater than 2 preferably represent from 1 to 10% by weight relative to the total weight of the composition.

In the particular case of retinoides, and still more particularly in the case of isotretinoin, this quantity preferably varies between 1 and 2.5% by weight relative to the total weight of the composition. Thus, as seen above, when the pharmaceutical composition in accordance with the invention contains a retinoide, then the presence of an oily phase, although possible, is nevertheless not essential.

In the particular case of hypolipidemic agents, and still more particularly in the case of fenofibrate, this quantity preferably varies between 5 and 10% by weight relative to the total weight of the composition.

In the particular case of steroid hormones, and still more particularly in the case of progesterone, this quantity preferably varies between 3 and 7% by weight relative to the total weight of the composition.

Among the surfactants having an HLB of less than 16, there may be mentioned in particular the surfactants which behave as good solvents for the AP to be formulated, among which are polyglycolized C₈-C₁₈ glycerides, in other words macrogol glycerides of C₈-C₁₀ chain fatty acids, such as caprylocapric macrogol glycerides such as for example the mixture of mono-, di- and triglycerides and of mono- and diesters of polyethylene glycol marketed under the trademark Labrasol® (HLB =14) by the company Gattefosse, and the oleic esters of polyglycerol having an HLB=10 such as for example the product marketed under the trademark Plurol® oleic by the company Gattefosse or the mixture of polyglycolized glycerides of C₈-C₁₈ fatty acids marketed under the trademark Gelucire® by Gattefosse, including Gelucire 44/14, or lauric macrogol glycerides. There may also be mentioned polysorbates, in other words sorbimacrogol—or polyethylene glycols (PEG)-esters of C₁₂-C₁₈ fatty acids such as hydrogenated ricinoleic, stearic, palmitic, oleic and lauric acids and their derivatives, marketed under the trademarks Ablunol® (Taiwan Surf), Aldosperse® (Lonza), Arlacel® (ICI), Crillet® (Croda), Drewmulse® (Stepan Food Ingredients), Ethylan® (Akcros), Emulpharma® (Respharma), Eumulgin® (Henkel), Montanox® (Seppic), Nikkol® (Nikko Chem. Co), Nissan Nonion® (Nippon Oils & Fats), Sorbilene® (Auschem), Sorgen® TW (Dai-ichi Kogyo Seiyaku) and Tween® (BASF); macrogol and propylene glycol esters of C₈-C₁₈ fatty acids (hydrogenated ricinoleic, stearic, capric, caprylic), marketed under the trademarks Captex® (Hüls), Cremophor® (BASF), Drewmulse® (Stepan Food Ingredients), DUB CAPS and DUB 810 (Stéarineries Dubois), Eumulgin® (Henkel) and Tagat (Goldschmidt); the macrogol-glyceride esters of C₁₂-C₁₈ fatty acids (hydrogenated ricinoleic, stearic, palmitic, oleic, lauric), marketed under the trademarks Akolip® (Karlshamns), Capmul® (Abitec), Cremophor® (BASF), Emulpharma® (Respharma), Ethylan® (Akcros), Eumulgin® (Henkel), Etocas® (Croda), Myrj® (ICI), Nikkol® (Nikko Chem. Co) and Tagat® (Goldschmidt); the polyglyceric esters of C₁₂-C₁₈ fatty acids (isostearic, lauric, oleic or stearic), marketed under the trademarks Caprol® (Abitec), Drewpol® (Stepan Food Ingredients) and Nikkol Decaglyn® (Nikko Chem. Co); and mixtures thereof.

These surfactants represent at least 50% and preferably from 70 to 85% of the total weight of the composition.

For the purposes of the present invention, a cosurfactant is considered as a good solvent for the active principle(s) present in the pharmaceutical composition in accordance with the invention, when it makes it possible, during dissolution trials, to solubilize enough active principle(s) while being compatible with the formulation of the finished product. For example, isotretinoin is 3% soluble in monoethyl ether of diethylene glycol (marketed under the trademark Transcutol® by Gattefosse; fenofibrate is 5% soluble in the same solvent; progesterone is 5% soluble in propylene glycol monocaprylate (marketed under the trademark Capryol by Gattefosse.

In accordance with the invention, the Ss and the CoSs are preferably nonionic compounds.

Among the CoSs which can be used in the pharmaceutical composition in accordance with the invention, there are preferably used the CoSs which behave as good solvents for the AP to be formulated and among which there may be mentioned in particular monoethyl ether of diethylene glycol (MEDG) corresponding, for example, to the product sold under the trademark Transcutol® by the company Gattefosse. There may also be mentioned the CoSs involved as solvent for the AP to be formulated, such as N-methyl-2-pyrrolidone or Pharmasolve® (ISP), the triester of glycerol and acetic acid or Triacetin® (Aldrich), dimethyl isosorbate (Aldrich), polyethylene glycols (PEG) such as PEG-400 and PEG-600 (in other words PEG-8 and PEG-12) and the products marketed for example under the names Carbowax® (Union Carbide), Lipoxol® (Hüls), Pluracol® (BASF), and the alcohols and glycols used as solvents or cosolvents, ethanol, isopropanol, glycerol, propylene glycol, butylene glycol, glycofurol and sorbitol; mono- and diesters of propylene glycol and of caprylic, capric and lauric fatty acids, marketed under the trademarks Labrafac® PG, Capryol® and Lauroglycol® (Gattefosse) ; mono- and diglycerides of caprylic, capric, lauric, oleic, stearic fatty acids marketed under the trademarks Akoline® (Karlshamns), Capmul® (Abitec), Drewmulse® (Stepan Food Ingredients), DUB GMS (Stéarineries Dubois), Imwitor® (Hüls), Maisine® and Peceol® (Gattefosse); and mixtures thereof.

These CoSs preferably represent from 5% to 20% by weight relative to the total weight of the composition.

When the pharmaceutical composition in accordance with the invention contains a retinoid, and in particular isotretinoin, as AP, then the CoS concentration is more preferably between 10 and 15% by weight relative to the total weight of the composition.

When the pharmaceutical composition in accordance with the invention contains a hypolipidemic agent such as fenofibrate, as AP, then the CoS concentration is more preferably between 5 and 10% by weight relative to the total weight of the composition.

According to the invention, the lipophilic phase preferably has an HLB of less than or equal to 4, is liquid at room temperature and is preferably chosen from fatty acid esters, in particular macrogol (or polyethylene glycol) glycerides, in other words polyglycolized glycerides of fatty acids such as for example PEG-6 glyceryl monooleate having an HLB=3 sold under the trademark Labrafil® M 1944 CS by the company Gattefosse, PEG-6 glyceryl linoleate having an HLB=4 sold under the trademark Labrafil® M 2125 CS by the company Gattefosse; sorbitan esters of saturated or unsaturated fatty acids such as lauric, oleic, stearic, palmitic, sesquioleic acids and their derivatives, marketed under the trademarks Arlacel® (ICI), Crill® (Croda), Drewmulse® (Stepan Food Ingredients), Ethylan® (Akcros), Glycomul® (Lonza), Kemester® (Witco Oleo-Surf), Montane® (Seppic), Nikkol® (Nikko Chem Co), Nissan Nonion® (Nippon Oils & Fats), Sorbirol® (Auschem), Sorgen® TW (Dai-ichi Kogyo Seiyaku) and Span (ICI); glycerol, propylene or butylene glycol esters of fatty acids marketed under the trademarks Arlacel® (ICI), Capmul® (Abitec), Drewmulse® (Stepan Food Ingredients), DUB GMS (Stéarineries Dubois), Imwitor® and Miglyol® (Hüls), Maisine®, Olicine® and Peceol® (Gattefosse); medium chain triglycerides of caprylic, capric and lauric fatty acids such as the products marketed under the trademarks Akomed® (Karlshamns), Captex® (Abitec), Crodamol® (Croda), DUB MCT (Stéarineries Dubois), Imwitor® and Miglyol® (Hüls), Labrafac® CC (Gattefosse), Neobee® (Stepan Food Ingredients); and mixtures thereof.

According to a preferred embodiment of the invention, and when the AP is a retinoid such as isotretinoin, then the pharmaceutical composition contains a lipophilic phase in a proportion preferably of between 3 and 4.5% by weight relative to the total weight of the composition.

The oily phase may be chosen from oils of natural and synthetic origin.

Among the oils of natural origin, there may be mentioned in particular almond, peanut, rapeseed, cotton seed, linseed, corn, olive, borage, evening primrose, fish, palm, palm kernel, grapeseed, sesame, soybean and sunflower oils, and the like. These oils may be from the first pressing, refined or interesterified, such as the oils marketed under the trademarks Akofine®, Akosoft®, Akosol® (Karlshamns), Myverol®, Myvacet® (Eastman) and Neobee® (Stepan Food Ingredients).

Among the oils of synthetic origin, the oils having an HLB value of less than or equal to 5, and still more particularly of less than or equal to 3, are preferred, among which there may be mentioned in particular the products marketed under the trademarks Captex® (Abitec), Crodamol® (Croda), DUB 810 PG (Stéarineries Dubois), Neobee® (Stepan Food Ingredients) and Labrafac® (Gattefosse).

When the oily phase consists of a synthetic oil, it may, according to the invention, have a very low HLB, of the order of 1 to 3, preferably chosen from the fatty acid esters sold under the name Labrafac®, such as Labrafac® PG, Labrafac® CC or Labrafac® lipophilic (Gattefosse), and mixtures thereof.

According to a preferred embodiment of the invention, and when the AP is a hypolipidemic agent such as fenofibrate, then the pharmaceutical composition contains an oily phase in a proportion preferably of between 2 and 12% by weight.

In the case where the composition requires it, it may prove useful to incorporate preservatives either into the lipid solution or into the envelope of the capsule such as for example: butylated hydroxyanisole (BHA), butylated hydroxytoluene (BHT), group E vitamins or tocopherols, ethylenediaminetetraacetic acid (EDTA) or its salts, methyl- or propylparabens, salts of para-hydroxybenzoic acid derivatives, etc.

According to an advantageous embodiment of the invention and when the pharmaceutical composition contains a retinoid, and in particular isotretinoin, then the composition contains at least one group E vitamin, preferably vitamin E.

The presence of vitamin E is indeed advantageous insofar as, in addition to its antioxidant effect toward isotretinoin, it also makes it possible to avoid the formation of crystals during storage of the compositions in accordance with the invention at low temperature.

The pharmaceutical composition in accordance with the invention may also contain one or more thickeners chosen from celluloses, waxes, acrylic polymers and gums. This thickening agent is preferably chosen from celluloses such as carboxymethylcellulose, hydroxypropylmethylcellulose, hydroxymethylcellulose and methylcellulose.

As thickening agents, there may also be used the surfactants described above, in particular esters of fatty acids and of polyethylene glycol.

According to an advantageous embodiment of the invention, and when the pharmaceutical composition contains a retinoid such as isotretinoin, then it also preferably contains at least one thickening agent.

The pharmaceutical composition in accordance with the invention may be packaged in hard gelatin capsules or soft gelatin capsules, for example made of gelatin, which, after ingestion orally and disintegration, will release the pharmaceutical composition in accordance with the invention which will spontaneously form a microemulsion upon contact with physiological fluid.

The pharmaceutical compositions in accordance with the invention may be prepared according to a method consisting:

in a first step, of dissolving the AP to be formulated in its appropriate solvent which is the CoS, optionally in the presence of a cosolvent or a surfactant or of the lipophilic phase and optionally of the oily phase when it is present;

and then in a second step, of incorporating this solution, with stirring and/or homogenization, into the liquid or liquefied surfactant, in which case the oily phase may be mixed beforehand, according to one variant of this same method, at a temperature which makes it possible to obtain a homogeneous solution, and then

in a third step, after returning to room temperature, of distributing the solution thus obtained into hard gelatin capsules or soft gelatin capsules, the quantity of solution incorporated being calculated according to the required unit dose of AP.

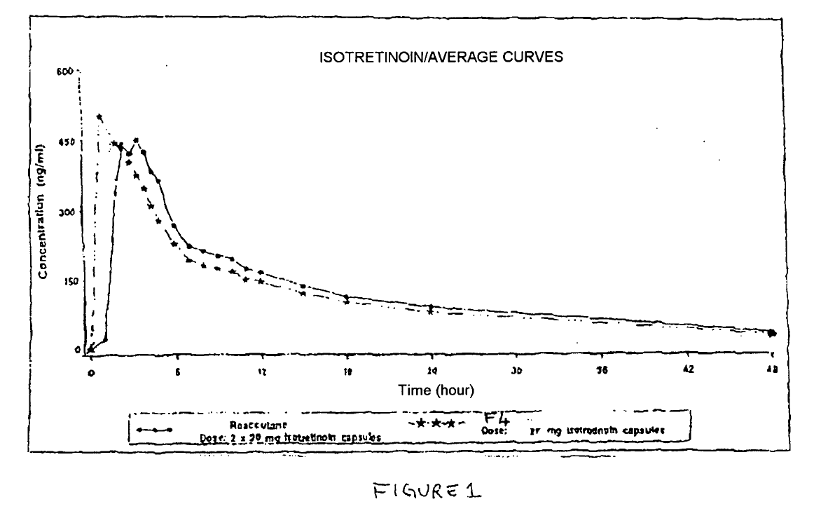

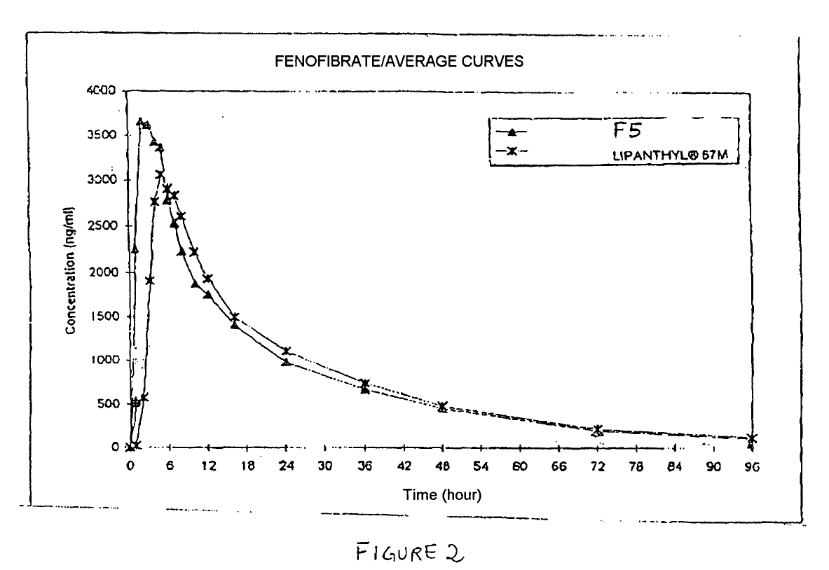

In addition to the preceding features, the invention further comprises other features which will emerge from the description which follows, which refers to an example relating to a comparative study of the stability of a composition in accordance with the invention compared with a composition as described in the prior art, to an example relating to a comparative study of the permeability of Caco-2 cells to isotretinoin in various formulations, to an example relating to the study of the bioavailability of the isotretinoin formulated in accordance with the invention compared with the commercial presentation ROACCUTANE®, to an example of a formulation based on fenofibrate, to an example relating to the study of the bioavailability of the fenofibrate formulated in accordance with the invention compared with the commercial presentation LIPANTHYL® 67M, to an example of a formulation based on progesterone, and to the accompanying FIGS. 1 and 2 in which:

FIG. 1 represents the bioavailability of the isotretinoin formulated in accordance with the invention compared with that of the commercial product ROACCUTANE®;

FIG. 2 represents the bioavailability of the fenofibrate formulated in accordance with the invention compared with that of the commercial product LIPANTHYL® 67M.

It should be clearly understood however that these examples are given solely by way of illustration of the subject of the invention, and do not constitute in any manner a limitation thereto.

### EXAMPLE 1

### Comparative Study of the Stability and Homogentity of Compositions Based on Isotretinoin

1) Pharmaceutical Compositions Prepared

Two pharmaceutical compositions based on isotretinoin containing the ingredients presented in Table I below were prepared, the percentages indicated are by weight:

TABLE I

| | F1 in accordance | F2 not forming part |
|---|---|---|
| Composition | with the invention | of the invention |
| Isotretinoin | 1.4% | 1.4% |
| Surfactant | Labrasol ® | Labrasol ® |
| | (HLB = 14): 81% | (HLB = 14): 9.7% |
| Cosurfactant | Transcutol ®: 13.6% | Capryol ® (HLB = 5): |
| | | 13.9% |
| Lipophilic phase | Labrafil ® M 1944 CS | Gelucire ® 44/14 |
| | (HLB = 3): 4% | (HLB = 14): 75% |

The CAPRYOL® used in the composition F2 is a propylene glycol monocaprylate containing 60% of monoesters.

2) Study of the Stability and Homogeneity of the Compositions F1 and F2

It was observed that the composition F1 in accordance with the invention, that is to say containing less than 5% of a lipophilic phase having a low HLB, was liquid at room temperature and led, in the presence of a hydrophilic phase, to the formation of a fine microemulsion (120 nm), which was stable at 25° C., which is a temperature which discriminates in relation to the constituents of the self-emulsifying system, and which was homogeneous.

On the other hand, the composition F2 not forming part of the invention, because it contains a large quantity of lipophilic phase (75%) and having a high HLB (HLB=14), leads to a semisolid formulation at room temperature, which is unstable and leads, in the presence of a hydrophilic phase, to a nonhomogeneous micellar solution in the form of microdroplets, composed of two different populations of micelles in terms of size: on average 112 nm (33%) and 900 nm (67%).

### EXAMPLE 2

### Comparative Study of the Permeability to Isotretinoin as a Function of its Formulation

In order to carry out this study, the pharmaceutical composition F1 in accordance with the invention and as prepared above in Example 1 was compared with a composition F3 consisting of a solution of isotretinoin alone, at 1.4% in dimethyl sulfoxide (DMSO), and to the commercial formulation of isotretinoin sold under the trademark ROACCUTANE® containing isotretinoin in a mixture of excipients composed of yellow beeswax, hydrogenated and nonhydrogenated soybean oils, and partially hydrogenated vegetable oil.

The permeability study was carried out on intestinal epithelial cells Caco-2, according to the modalities described in the articles by I J Hidalgo et al., “Characterization of the human colon carcinoma cell line (Caco-2) as a model system for intestinal epithelial permeability”, Gastroenterology, 1989, 96, 736-749 and P Artursson et al., “Correlation between oral drug absorption in humans and apparent drug permeability coefficients in human intestinal epithelial (Caco-2) cells”, Biochem. Biophys. Res. Commun., 1991, 175, 880-885.

Indeed, the intestinal absorption may be studied in vitro by means of differentiated cell cultures.

The intestinal cell line Caco-2 (derived from human colorectal carcinoma) develops the morphological characteristics of normal enterocytes (columnar epithelium of the wall of the small intestine).

When they are cultured on polycarbonate membrane, the Caco-2 cells form a monocellular layer of polarized enterocytes.

They are characterized as a representative model of the transport system of the epithelium of the small intestine, and from a morphological point of view and in terms of permeability toward solutes which are not the subject of a membrane permeation (I J Hidalgo et al., cited above).

The study of the passive absorption of AP across the intestinal epithelium, using the Caco-2 model, was validated with the aid of various APs.

This made it possible to establish a correlation between their absorption per os and the calculated apparent coefficient of permeability using the Caco-2 cellular model (P Artursson et al., cited above).

The membrane integrity of the Caco-2 cells was also studied. This integrity was monitored by virtue of the incorporation, into the working solution, of a carbon 14-labeled mannitol solution.

Thus, this tracer makes it possible to verify that the passage across the membrane and not outside went well. Indeed, the permeability to mannitol for all the solutions tested is comparable to the control permeability for mannitol.

The results obtained are presented in Table II below:

TABLE II

| | F1 in | | |
|---|---|---|---|
| | accordance | F3 (solution of | |
| | with the | isotretinoin | |
| Compositions | invention | alone) | ROACCUTANE ® |
| Permeability to | 2.14 | 3.79 | <0.1 |
| isotretinoin | | | |
| (10⁻⁶ cm/s) | | | |
| Integrity of | preserved | preserved | preserved |
| the membrane | | | |

These results show that the membrane integrity was preserved for all the compounds tested.

They also show that the pharmaceutical composition F1, in accordance with the invention, makes it possible to increase the permeability of the Caco-2 cells to isotretinoin and, consequently, the bioavailability of this active principle.

The good results of permeability of the composition F3 containing isotretinoin alone is explained by the fact that this active principle was completely dissolved in DMSO; however, this excellent solvent for organic substances cannot be used as it is in pharmaceutical preparations for toxicity considerations.

### EXAMPLE 3

### Comparative Study of the Bioavailability of Isotretinoin as a Function of its Formulation

In order to carry out this study, the following pharmaceutical composition F4 in accordance with the invention was prepared:

| | |
|---|---|
| Active principle: isotretinoin | 2% |
| S: Labrasol ® (HLB = 14) | 80.5% |
| Co-S: Transcutol ® | 13.5% |
| Lipophilic phase: Labrafil ® M1944 (HLB = 3) | 4% |

This pharmaceutical composition F4 was packaged in hard gelatin capsules each containing 9 mg of isotretinoin.

The bioavailability study was carried out according to a protocol for administering a single dose of each of the treatments: the test formula and the reference formula are administered according to a criss-cross and randomized order.

The isotretinoin formulated according to the pharmaceutical composition F4 was compared with that of the commercial formulation of isotretinoin sold under the trademark ROACCUTANE® and as described above in Example 2.

In order to observe the customary mode of administration and dosage of the reference proprietary product ROACCUTANE®, the two products were administered, during meals, at the rate of the single dose of 3 soft gelatin capsules of the composition F4 and two soft gelatin capsules of ROACCUTANE® at 20 mg.

The results obtained are presented in FIG. 1 which correspond to the plasma concentration of isotretinoin in ng/ml as a function of the time in hour.

At equal doses of AP isotretinoin, these results show a suprabioavailability of 37.5% for the composition F4, in terms of intensity of absorption, according to the comparative results of the areas under the total plasma concentration curves (AUC) and the plasma concentration peaks Cₘₐₓ (result greater than 48%, with in particular a lower variability of the pharmacokinetic response in the case of the composition F4).

These results also demonstrate a bioequivalence between the administered dose of 27 mg of isotretinoin (composition F4 in accordance with the invention) and the dose which is nevertheless greater than 40 mg of isotretinoin of ROACCUTANE®.

In terms of rate of absorption, these results also show that the composition F4 in accordance with the invention is more rapid than the reference composition ROACCUTANE®, with an earlier plasma concentration peak, expressed by the parameter Tₘₐₓ, which is one hour on average for the composition F4, against 3 hours on average for ROACCUTANE®.

### EXAMPLE 4

### Pharmaceutical Composition Based on Fenofibrate

The following pharmaceutical composition F5, in accordance with the invention, was prepared:

| | |
|---|---|
| Active principle: fenofibrate | 8.1% |
| S: Labrasol ® (HLB = 14) | 74.0% |
| Co-S: Transcutol ® | 6.25% |
| Lipophilic phase: Labrafil ® M1944 | 0.8% |
| Oily phase: Labrafac ® PG (HLB = 1) | 6.25% |
| Oily phase: Labrafac ® CC (HLB = 1) | 4.6% |

In the presence of an aqueous phase, this composition spontaneously led to a stable and fine microemulsion in which the fenofibrate was perfectly dissolved.

### EXAMPLE 5

### Comparative Study of the Bioavailability of Fenofibrate as a Function of its Formulation

This study was carried out under the same conditions as those described above in Example 3, in order to compare the bioavailability of the fenofibrate formulated in accordance with the invention (composition F5 as described above in Example 4) to the commercial fenofibrate formulation (micronized form) sold under the trademark LIPANTHYL® 67M.

The composition F5 was packaged in hard gelatin capsules each containing 66 mg of fenofibrate.

The composition F5 and LIPANTHYL® was administered during a meal at the rate of 1 hard gelatin capsule.

The results obtained are presented in FIG. 2, which correspond to the plasma concentration of fenofibric acid in ng/ml as a function of the time in hour.

In terms of rate of absorption, the composition F5 is more rapid than LIPANTHYL®, with an earlier plasma concentration peak, expressed by the parameter Tₘₐₓ, which is 2.5 hours on average for the composition F5 against 5.83 hours on average for LIPANTHYL®.

This bioequivalence between the composition F5 and the dose of 67 mg of the formulation LIPANTHYL® is particularly advantageous insofar as the composition F5 is found to be bioequivalent to the formulation LIPANTHYL®, which is itself suprabioavailable compared to the previously marketed nonmicronized form.

Consequently, the self-emulsifying and micelle-forming composition F5 in accordance with the invention makes it possible to obtain the maximum bioavailability of the fenofibrate while applying to this active principle a galenic route of preparation which is completely different from micronization, whereas, up until now, only the micronization of fenofibrate had made it possible to improve its bioavailability.

### EXAMPLE 6

### Pharmaceutical Composition Based on Progesterone

The following pharmaceutical composition F6 in accordance with the invention was prepared:

| | |
|---|---|
| Active principle: Progesterone | 5% |
| S: Labrasol ® (HLB = 14) | 8.5% |
| S: Gelucire ® 44/14 (HLB = 14) | 65% |
| Co-S: Capryol ® PGMC (HLB = 5 to 6) | 17.5% |
| Lipophilic phase: Labrafil ® M1944 | 2% |
| Oily phase: Labrafac ® CC (HLB = 1) | 2% |

This composition is essentially distinguishable from the prior art composition F2, which is not stable after a few months, by the fact that it contains 4% of oily phase. It spontaneously leads, in the presence of an aqueous phase, to a stable and fine microemulsion in which the progesterone is perfectly dissolved.

Indeed, it should be noted that the lipophilicity of the AP plays an important role in the equilibrium of the system. The addition of the oily phase makes it possible to better solubilize the progesterone and it is this oil which will be micellized and which makes it possible to significantly improve the stability.

1. A self-micro emulsifying pharmaceutical composition for oral use comprising:
at least one lipophilic active principle,
at least one surfactant having a hydrophilic/lipophilic balance of less than 16,
at least one cosurfactant,
at least one lipophilic phase, characterized in that:
the lipophilic active principle(s) have a log P greater than 2,
the surfactant(s) represent at least 50% by weight of the total weight of said composition,
the cosurfactant(s) are chosen from the good solvents for said active principle(s),
the lipophilic phase is optionally surface-active and represents less than 0.5 to 4.5% by weight of the total weight of said composition and has an HLB of less than or equal to 6, and
when the active principle is different from a retinoid, then said composition additionally comprises a nonsurfactant oily phase representing from 1 to 12% of the total weight of said composition.

2. The composition as claimed in claim 1, characterized in that the active principle(s) have a log P greater than 4.

3. The composition as claimed in claim 1 or 2, characterized in that the active principle(s) are chosen from retinoids, hypolipidemic agents, steroid hormones, steroid anti-inflammatories, nonsteroid anti-inflammatories (NSAIDs), antiretrovirals, protease inhibitors (“navirs”), antiacids, proton pump inhibitors, antiemetics, fat-soluble vitamins, cardiovascular system drugs, platelet aggregation inhibitors, cancer drugs, certain plant extracts and their isolated or derived APs, immunosuppressants, central nervous system drugs, antimigraines, antibiotics, antifungals and antiparasitics.

4. The composition as claimed in claim 3, characterized in that the active principle(s) are chosen from retinoids, hypolipidemic agents and steroid hormones.

5. The composition as claimed in claim 3 or 4, characterized in that it contains isotretinoin in a quantity of between 1 and 2.5% by weight relative to the total weight of the composition.

6. The composition as claimed in claim 3 or 4, characterized in that it contains fenofibrate in a quantity of between 5 and 10% by weight relative to the total weight of the composition.

7. The composition as claimed in claim 2 or 3, characterized in that it contains progesterone in a quantity of between 3 and 7% by weight relative to the total weight of the composition.

8. The composition as claimed in any one of the preceding claims, characterized in that the surfactant(s) are chosen from polyglycolized C₈-C₁₈ glycerides, polysorbates or polyethylene glycols (PEG)-esters of C₁₂-C₁₈ fatty acids, macrogol and propylene glycol esters of C₈-C₁₈ fatty acids, macrogol-glyceride esters of C₁₂-C₁₈ fatty acids, polyglyceric esters of C₁₂-C₁₈ fatty acids, and mixtures thereof.

9. The composition as claimed in any one of the preceding claims, characterized in that the surfactant(s) represent from 70 to 85% of the total weight of the composition.

10. The composition as claimed in any one of the preceding claims, characterized in that the cosurfactant(s) are chosen from diethylene glycol monoethyl ether; N-methyl-2-pyrrolidone; the triester of glycerol and acetic acid; dimethyl isosorbate; polyethylene glycols; alcohols and glycols; mono- and diesters of propylene glycol and of caprylic, capric, lauric fatty acids; mono- and diglycerides of caprylic, capric, lauric, oleic, stearic fatty acids; and mixtures thereof.

11. The composition as claimed in any one of the preceding claims, characterized in that the cosurfactant(s) represent from 5% to 20% of the total weight of the composition.

12. The composition as claimed in claim 5, characterized in that the cosurfactant concentration is between 10 and 15% of the total weight of the composition.

13. The composition as claimed in claim 6, characterized in that the CoS concentration is between 5 and 10% of the total weight of the composition.

14. The composition as claimed in any one of the preceding claims, characterized in that the lipophilic phase has an HLB of less than or equal to 4, and is chosen from fatty acid esters; sorbitan esters of saturated or unsaturated fatty acids and their derivatives; glycerol, propylene or butylene glycol esters of fatty acids; medium chain triglycerides of caprylic, capric or lauric fatty acids; and mixtures thereof.

15. The composition as claimed in claim 14, characterized in that the fatty acid esters are chosen from macrogol (or polyethylene glycol) glycerides, in other words polyglycolized glycerides of fatty acids.

16. The composition as claimed in claim 5, characterized in that it contains a lipophilic phase in a proportion of between 3 and 4.5% by weight relative to the total weight of the composition.

17. The composition as claimed in claim 5, characterized in that it contains a nonsurfactant oily phase representing from 1 to 12% of the total weight of said composition and at least one thickening agent.

18. The composition as claimed in any one of the preceding claims, characterized in that the oily phase is chosen from oils of natural and synthetic origin.

19. The composition as claimed in claim 18, characterized in that the natural oils are chosen from almond, peanut, rapeseed, cotton seed, linseed, corn, olive, borage, evening primrose, fish, palm, palm kernel, grapeseed, sesame, soybean and sunflower oils.

20. The composition as claimed in claim 18, characterized in that the synthetic oils are chosen from fatty acid esters whose HLB value is between 1 and 3.

21. The composition as claimed in claim 6, characterized in that it contains an oily phase in a proportion of between 2 and 15% by weight.

22. The composition as claimed in any one of the preceding claims, characterized in that it leads, in the presence of a hydrophilic phase, to the formation of a microemulsion in which the size of the micelles is less than 500 nm, and more particularly between 1 and 200 nm.

23. The composition as claimed in any one of the preceding claims, characterized in that it is packaged in hard gelatin capsules or in soft gelatin capsules.
